(19) United States
(12) Patent Application Publication
(10) Pub. No.: US 2012/0022359 A1
(43) Pub. Date: Jan. 26, 2012

(54) CAPSULE MEDICAL DEVICE GUIDANCE SYSTEM

(75) Inventors: Hironao KAWANO, Tokyo (JP); Henrik KELLER, Luitpoldstrasse (DE)

(73) Assignee: OLYMPUS MEDICAL SYSTEMS CORP., Tokyo (JP)

(21) Appl. No.: 13/160,824

(22) Filed: Jun. 15, 2011

Related U.S. Application Data
(63) Continuation of application No. PCT/JP2010/064282, filed on Aug. 24, 2010.

(30) Foreign Application Priority Data
Nov. 10, 2009 (JP) 2009-257480

(51) Int. Cl. A61B 5/05

(52) U.S. Cl. 600/409

(57) ABSTRACT

A capsule medical device guidance system includes a capsule medical device including a magnetic field responding unit and introduced into a subject, a magnetic field generator that generates a magnetic field for the responding unit to guide the device; an operation input unit for inputting operation information for magnetically guiding the device; a storage unit that stores position/posture information about position or posture of the device in an guidance region; an input unit for inputting setting instruction information for setting the position/posture information as a mark indicating a position to which the device is returned in the region, and return instruction information for returning the device to the mark; and a control unit that controls the generator to guide the device based on the operation information, and makes the generator generate the magnetic field to the mark based on the stored position/posture information in response to the return instruction information.

### CROSS-REFERENCES TO RELATED APPLICATIONS

This application is a continuation of PCT international application Ser. No. PCT/JP2010/064282 filed on Aug. 24, 2010 which designates the United States, incorporated herein by reference, and which claims the benefit of priority from Japanese Patent Application No. 2009-257480, filed on Nov. 10, 2009, incorporated herein by reference.

### BACKGROUND OF THE INVENTION

1. Field of the Invention

The present invention relates to a capsule medical device guidance system which guides a capsule medical device introduced into a subject.

2. Description of the Related Art

Conventionally, in the field of an endoscope, a capsule medical device has been introduced which has an image capturing function and a wireless communication function inside a capsule-shaped casing formed in a size which can be introduced in the digestive tract of a subject such as a patient. The capsule medical device is swallowed from the mouth of the subject and then moves in the digestive tract by peristaltic motion. In a period in which the capsule medical device is introduced inside the digestive tract of the subject and is excreted from the subject, the capsule medical device sequentially obtains images (also referred to “in-vivo images”) of internal organs of this subject, and sequentially transmits by radio the obtained in-vivo images to a receiving device outside the subject.

Each in-vivo image captured by this capsule medical device is taken in an image display device through the receiving device. The image display device displays each in-vivo image which is taken in, on a display as still images or movies. Users such as doctors or nurses observe each in-vivo image of the subject displayed on the image display device, and examine the internal organs of the subject by observing each in-vivo image.

By contrast with this, in recent years, a capsule medical device guidance system is proposed which guides the capsule medical device inside the subject by means of a magnetic force (hereinafter “magnetic guidance”). Generally, in the capsule medical device guidance system, the capsule medical device further includes a permanent magnet inside the capsule-shaped casing, and the image display device displays in real time each in-vivo image sequentially captured by the capsule medical device inside the subject. The capsule medical guidance system applies the magnetic field to the capsule medical device inside the subject, and magnetically guides the capsule medical device inside the subject, to a desired position by means of a magnetic force of this applied magnetic field. The user performs an operation of magnetically guiding the capsule medical device using the operating unit in the capsule medical device guidance system while referring to in-vivo images displayed on this image display device.

To observe internal organs having relatively a large space such as the stomach or large intestine, there is a capsule endoscope as this capsule medical device which sequentially captures in-vivo images in a state where the capsule endoscope floats in a liquid with a specific gravity which allows the capsule endoscope to float in a liquid injected inside the organs. Further, to intensively examine the internal organs having relatively a large space such as the stomach, there are cases where the subject swallows a liquid for expanding the internal organ (more specifically, folds of an inner wall of an organ), and a capsule endoscope with a specific gravity smaller than this liquid (for example, International Publication No. 2007/077922). In this case, the capsule endoscope sequentially captures images of the internal organs expanded by this liquid, while floating at a liquid level in a mode of taking a predetermined posture (for example, a vertical posture such that the center axis in the longitudinal direction of the capsule endoscope and the liquid level are nearly vertical) in the internal organs such as the stomach. The capsule endoscope can capture images of the internal organs in a wide range by moving in a desired direction in a state where the capsule endoscope floats at the liquid level of the internal organ.

### SUMMARY OF THE INVENTION

A capsule medical device guidance system according to an aspect of the present invention includes a capsule medical device to be introduced into a subject, the capsule medical device including a magnetic field responding unit; a magnetic field generator that generates a magnetic field for the magnetic field responding unit to guide the capsule medical device; an operation input unit for inputting operation information for magnetically guiding the capsule medical device; a storage unit that stores position/posture information about a position or a posture of the capsule medical device, in an guidance region in which the magnetic field generator allows the capsule medical device to be guided; an instruction information input unit for inputting setting instruction information for setting the position/posture information as a mark indicating a position to which the capsule medical device is returned in the guidance region, and return instruction information for returning the capsule medical device to the mark; and a control unit that controls the magnetic field generator to guide the capsule medical device in accordance with the operation information input through the operation input unit, and makes the magnetic field generator generate a magnetic field for guiding the capsule medical device to the mark based on position/posture information stored in the storage unit when the return instruction information is input through the instruction information input unit.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic view illustrating an entire configuration of a capsule medical device guidance system according to a first embodiment;

FIG. 2 is a sectional schematic view illustrating a configuration example of a capsule endoscope illustrated in FIG. 1;

FIG. 3 is a view describing a peak magnetic field generated by a magnetic field generator illustrated in FIG. 1;

FIG. 4 is a view describing a uniform gradient magnetic field generated by a magnetic field generator illustrated in FIG. 1;

FIG. 5 is a view illustrating an example of an operation input unit illustrated in FIG. 1;

FIG. 6 is a view describing movement of a capsule endoscope illustrated in FIG. 1;

FIG. 7 is a view illustrating a magnetic field generating area from above;

FIG. 8 is a view describing movement of a capsule endoscope illustrated in FIG. 1;

FIG. 9 is a schematic view illustrating an entire configuration of a capsule medical device guidance system according to a second embodiment;

FIG. 10 is a schematic view illustrating another entire configuration of a capsule medical device guidance system according to the second embodiment;

FIG. 11 is a conceptual diagram for describing a capsule endoscope illustrated in FIG. 10;

FIG. 12 is a schematic view illustrating another entire configuration of a capsule medical device guidance system according to the second embodiment;

FIG. 13 is a conceptual diagram for describing a capsule endoscope illustrated in FIG. 12;

FIG. 14 is a schematic view illustrating another entire configuration of a capsule medical device guidance system according to the second embodiment;

FIG. 15 is a conceptual diagram for describing a capsule endoscope illustrated in FIG. 14;

FIG. 16 is a conceptual diagram for describing another example of a capsule endoscope illustrated in FIG. 1;

FIG. 17A is a front view of an operation input unit for describing magnetic guidance of a capsule medical device which can be operated by the operation input unit;

FIG. 17B is a right side view of an operation input unit for describing magnetic guidance of a capsule medical device which can be operated by the operation input unit;

FIG. 17C is a view illustrating content of an operation of a capsule endoscope instructed by an operation of each component of the operation input unit;

FIG. 18 is a view illustrating another example of an operation input unit illustrated in FIG. 5;

FIG. 19 is a schematic view illustrating an example of each movement state of a table part of a bed and magnetic field generator forming a capsule medical device guidance system according to the present invention; and

FIG. 20 is a schematic view illustrating an example of a magnetic field generator of a capsule medical device guidance system according to the present invention.

### DETAILED DESCRIPTION OF THE PREFERRED EMBODIMENTS

Hereinafter, capsule medical device guidance systems according to embodiments of the present invention will be described using as examples capsule medical device systems which use as a body-insertable apparatus a capsule endoscope which is orally inserted in the subject and floats on a liquid stored in the stomach, small intestine or large intestine of the subject. Meanwhile, the capsule medical device guidance systems are not limited to this, and various body-insertable apparatuses may be used such as a monocular or multiocular capsule endoscope which obtains in-vivo images inside the subject by executing an operation of capturing images while moving in the lumen from, for example, the esophagus to the anus of the subject. In addition, the embodiments by no means limit the present invention. Further, the same parts will be assigned the same reference numerals in the drawings.

### First Embodiment

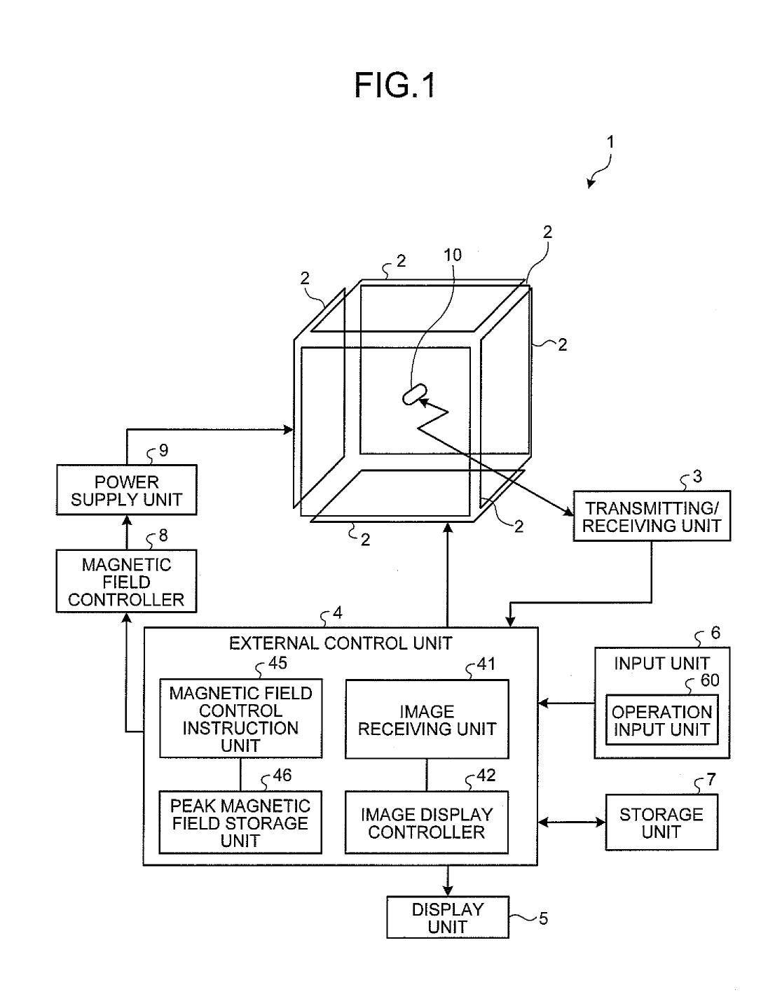

First, a first embodiment will be described. FIG. 1 is a schematic view illustrating an entire configuration of a capsule medical device guidance system according to the first embodiment of the present invention. As illustrated in FIG. 1, a capsule medical device guidance system 1 according to the first embodiment includes a capsule endoscope 10 which is a capsule medical device which is introduced in the body cavity of the subject by being swallowed from the mouth of the subject and communicates with an external device; a magnetic field generator 2 which is provided in the surrounding of the subject and which can provide a three-dimensional magnetic field; a transmitting/receiving unit 3 which performs wireless communication with the capsule endoscope 10 to receive a radio signal including an image captured by the capsule endoscope 10 and transmit an operation signal to the capsule endoscope 10; an external unit 4 which controls each component of the capsule medical device guidance system 1; a display unit 5 which outputs and displays an image captured by the capsule endoscope 10; an input unit 6 which inputs to the external unit 4 instruction information for instructing various operations of the capsule medical device guidance system 1 such as operation information for guiding the capsule endoscope 10 by means of the magnetism; a storage unit 7 which stores image information captured by the capsule endoscope 10; a magnetic field controller 8 which controls the magnetic field which works on the magnetic field generator 2; and a power supply unit 9 which supplies power to the magnetic field generator 2 according to control by the magnetic field controller 8.

The capsule endoscope 10 is a medical device of a capsule shape which obtains in-vivo images of the subject, and has a built-in image capturing function and wireless communication function. The capsule endoscope 10 is introduced inside the organ of the subject by, for example, oral ingestion. Then, the capsule endoscope 10 inside the subject moves inside the digestive tract, and is finally excreted from the subject. In a period in which the capsule endoscope 10 is introduced inside the subject and is excreted from the outside, the capsule endoscope 10 sequentially captures in-vivo images of the subject, and sequentially transmits by radio the obtained in-vivo images to the external transmitting/receiving unit 3. Further, the capsule endoscope 10 includes a built-in magnetic body such as a permanent magnet. The capsule endoscope 10 drifts in the liquid injected inside the organ of the subject (for example, inside the stomach), and is magnetically guided by the external magnetic field generator 2.

The magnetic field generator 2 magnetically guides the capsule endoscope 10 inside the subject. The magnetic field generator 2 is realized using, for example, a plurality of coils, and generates a guidance magnetic field using power supplied by the power supply unit 9. The magnetic field generator 2 applies this generated guidance magnetic field to the magnetic body inside the capsule endoscope 10, and magnetically captures the capsule endoscope 10 using the function of this guidance magnetic field. The magnetic field generator 2 changes a magnetic field direction of the guidance magnetic field which works on the capsule endoscope 10 inside the subject to control the three-dimensional posture of the capsule endoscope inside the subject. The magnetic field generator 2 can generate a so-called uniform magnetic field, and, in addition, a uniform gradient magnetic field and peak magnetic field. The magnetic field generator 2 generates a peak magnetic field which has a peak at a predetermined position on the horizontal plane.

The transmitting/receiving unit 3 has a plurality of antennas, and receives in-vivo images of the subject from the capsule endoscope 10 through a plurality of these antennas. The transmitting/receiving unit 3 sequentially receives radio signals from the capsule endoscope 10 through a plurality of these antennas. The transmitting/receiving unit 3 selects an antenna of the highest reception electric field intensity from a plurality of these antennas, and, for example, demodulates the radio signal received from the capsule endoscope 10 through this selected antenna. By this means, the transmitting/receiving unit 3 extracts image data, that is, in-vivo image data of the subject, of the capsule endoscope 10 from this radio signal. The transmitting/receiving unit 3 transmits the image signal including this extracted in-vivo image data, to the external unit 4.

The external unit 4 controls each operation of the magnetic field generator 2, the display unit 5, the storage unit 7 and the magnetic field controller 8, and controls an input and output of signals between these components. The external unit 4 includes an image receiving unit 41 which sequentially obtains in-vivo images sequentially received by the transmitting/receiving unit 3, and an image display controller 42 which displays the in-vivo images sequentially received by the transmitting/receiving unit 3 on the display unit 5 in real time. Further, the external unit 4 controls the storage unit 7 to store a group of in-vivo images of the subject obtained from the transmitting/receiving unit 3.

The external unit 4 includes a magnetic field control instruction unit 45 which instructs a magnetic field generating condition to the magnetic field controller 8 to guide the capsule endoscope 10 according to operation information inputted from the input unit 6, and a peak magnetic field storage unit 46 which stores the peak magnetic field generating condition as position/posture information related to the position or posture of the capsule endoscope 10.

The display unit 5 is realized using various displays such as a liquid crystal display, and displays various pieces of information instructed to be displayed by the external unit 4. More specifically, the display unit 5 displays, for example, a group of in-vivo images of the subject captured by the capsule endoscope 10, based on control of the image display controller 42 in the external unit 4. Further, the display unit 5 displays, for example, a reduced image of an in-vivo image selected or marked from the group of in-vivo images by the input operation of the input unit 6, patient information of the subject and examination information.

The input unit 6 is realized using an input device such as a keyboard or mouse, and inputs various pieces of information to the external unit 4 according to an input operation of an operator such as a doctor. The various pieces of information inputted from the input unit 6 to the external unit 4 include, for example, instruction information for instructing the external unit 4, patient information of the subject and examination information. In addition, patient information of the subject is specifying information for specifying the subject, and includes, for example, a patient name of the subject, patient ID, date of birth, sex and age. Further, examination information of the subject is specifying information for specifying an examination of observing the interior of the digestive tract by inserting the capsule endoscope 10 inside the digestive tract of the subject, and includes, for example, an examination ID and examination date. Further, the input unit 6 inputs operation information for performing the above operation of the magnetic field generator 2 to magnetically guide the capsule endoscope 10.

The input unit 6 has an operation input unit 60 which inputs to the external unit 4 operation information for guiding the capsule endoscope 10 using the magnetism, in, for example, the magnetic guiding direction or to a magnetic guiding position of the capsule endoscope 10 which is the target of a magnetic guiding operation. The operation input unit 60 employs a configuration including joy sticks, various buttons and various switches, and inputs operation information to the external unit 4 when these joy sticks are operated by the operator. Further, in addition to operation information, the operation input unit 60 inputs setting instruction information for instructing to set a mark in a guidance region of the capsule endoscope 10 and return instruction information for instructing the capsule endoscope 10 to return to the mark.

The storage unit 7 is realized using a storage medium which stores information in a rewritable state such as a flash memory or hard disk. The storage unit 7 stores various pieces of information which the external unit 4 instructs the storage unit 7 to store, and sends information which the external unit 4 instructs the storage unit 7 to read from the stored various pieces of information, to the external unit 4. In addition, these various pieces of information stored in the storage unit 7 include, for example, each image data of a group of in-vivo images of the subject captured by the capsule endoscope 10, in-vivo image data selected by the input operation of the input unit 6 from each in-vivo image displayed on the display unit 5, and input information of patient information of the subject from the input unit 6.

The magnetic field controller 8 controls the energization amount of the power supply unit 9 with respect to the magnetic field generator 2 based on instruction information instructed in the external unit 4, and, by controlling this power supply unit 9, controls the magnetic field generator 2 to generate the guidance magnetic field required for magnetically guiding the capsule endoscope 10 according to the magnetic guiding direction and magnetic guiding position based on this operation information.

The power supply unit 9 supplies power (for example, alternate current) required for generating the above guidance magnetic field, to the magnetic field generator 2 based on control by the external unit 4 and the magnetic field controller 8. In this case, the power supply unit 9 adequately supplies power required for each of a plurality of coils included in the magnetic field generator 2. In addition, the magnetic field direction and magnetic field intensity of the above guidance magnetic field of the magnetic field generator 2 are controlled according to the energization amount from the power supply unit 9 to each coil in the magnetic field generator 2.

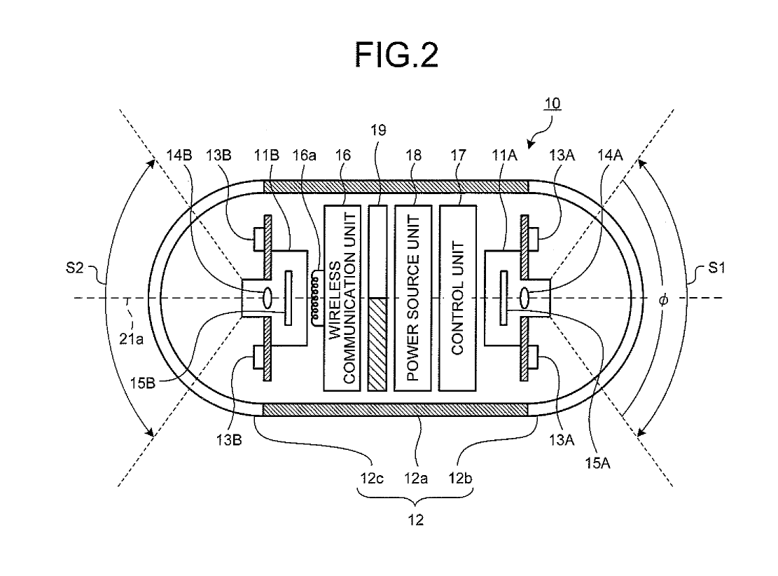

Next, the capsule endoscope 10 will be described. FIG. 2 is a sectional schematic view illustrating a configuration example of a capsule endoscope illustrated in FIG. 1. As illustrated in FIG. 2, the capsule endoscope 10 has a capsule-shaped casing 12 which is the exterior formed in a size which is easily introduced inside the organ of the subject, and imaging units 11A and 11B which capture images of the subject in respectively different imaging directions. Further, the capsule endoscope 10 includes a wireless communication unit 16 which transmits by radio to an outside each image captured by the imaging units 11A and 11B, a control unit 17 which controls each component of the capsule endoscope 10, and a power source unit 18 which supplies power to each component of the capsule endoscope 10. Further, the capsule endoscope 10 includes a permanent magnet 19 which enables the magnetic field generator 2 to perform magnetic guidance.

The capsule-shaped casing 12 is an outer casing formed in a size which can be introduced inside the organ of the subject, and is formed by covering aperture ends of both sides of a cylindrical casing 12a by dome-shaped casings 12b and 12c. The dome-shaped casings 12b and 12c are optical members of transparent dome shapes with respect to light of a band of a predetermined wavelength such as visible light. The cylindrical casing 12a is a colored casing which is nearly transparent with respect to visible light. As illustrated in FIG. 2, the capsule-shaped casing 12 formed with the cylindrical casing 12a and the dome-shaped casings 12b and 12c includes the imaging units 11A and 11B, the wireless communication unit 16, the control unit 17, the power source unit 18 and the permanent magnet 19.

The imaging units 11A and 11B capture images of respectively different imaging directions. More specifically, the imaging unit 11A includes an illuminating unit 13A such as a LED, an optical system 14A such as a condenser lens and an imaging element 15A such as a CMOS image sensor or CCD. The illuminating unit 13A emits illuminating light such as white light to an imaging field of view S1 of the imaging element 15A, and illuminates the subject (the inner wall of the organ on the imaging field of view S1 side inside the subject) in the imaging field of view S1 across the dome-shaped casing 12b. The optical system 14A condenses reflected light from this imaging field of view S1, on the imaging plane of the imaging element 15A, and forms the subject image of the imaging field of view S1 on the imaging plane of the imaging element 15A. The imaging element 15A receives reflected light from this imaging field of view S1 through the imaging plane, photoelectrically converts this received optical signal and captures subject images in this imaging field of view S1, that is, in-vivo images of the subject. The imaging unit 11B includes an illuminating unit 13B such as a LED, an optical system 14B such as a condenser lens and an imaging element 15B such as a CMOS image sensor or CCD. The illuminating unit 13B emits illuminating light such as white light to an imaging field of view S2 of the imaging element 15B, and illuminates the subject (the inner wall of the organ on the imaging field of view S2 side inside the subject) in the imaging field of view S2 across the dome-shaped casing 12c. The optical system 14B condenses reflected light from this imaging field of view S2, on the imaging plane of the imaging element 15B, and forms the subject image of the imaging field of view S2 on the imaging plane of the imaging element 15B. The imaging element 15B receives reflected light from this imaging field of view S2 through the imaging plane, photoelectrically converts this received optical signal and captures subject images in this field of view S2, that is, in-vivo images of the subject.

In addition, when the capsule endoscope 10 is a capsule medical device of a binocular type which captures images of the fore and rear of a long axis 21a direction as illustrated in FIG. 2, each optical axis of the imaging units 11A and 11B is nearly parallel to or virtually matches with the long axis 21a which is the center axis of the longitudinal direction of the capsule-shaped casing 12. Further, directions of the imaging field of views S1 and S2 of the imaging units 11A and 11B, that is, the imaging directions of the imaging units 11A and 11B are respectively opposite directions.

The wireless communication unit 16 includes an antenna 16a, and sequentially transmits by radio to the outside each image captured by the above imaging units 11A and 11B through the antenna 16a. More specifically, the wireless communication unit 16 obtains an image signal of an in-vivo image of the subject captured by the imaging unit 11A or the imaging unit 11B, from the control unit 17, and demodulates this obtained signal to generate a radio signal obtained by modulating the image signal. The wireless communication unit 16 transmits the radio signal to the external transmitting/receiving unit 3 through the antenna 16a.

The control unit 17 controls each operation of the imaging units 11A and 11B and the wireless communication unit 16 which are components of the capsule endoscope 10, and controls an input and output of signals between the components. More specifically, the control unit 17 makes the imaging element 15A capture an image of the subject in the imaging field of view S1 illuminated by the illuminating unit 13A, and makes the imaging element 15B capture an image of the subject in the imaging field of view S2 illuminated by the illuminating unit 13B. Further, the control unit 17 has a signal processing function of generating image signals. Every time the control unit 17 obtains in-vivo image data of the imaging field of view S1 from the imaging element 15A, the control unit 17 performs predetermined signal processing of this in-vivo image data, and generates an image signal including in-vivo image data of the imaging field of view S1. Similar to this, every time the control unit 17 obtains in-vivo image data of the imaging field of view S2 from the imaging element 15B, the control unit 17 performs predetermined signal processing of this in-vivo image data, and generates an image signal including in-vivo image data of the imaging field of view S2. The control unit 17 controls the wireless communication unit 16 to sequentially transmit by radio each image signal to the outside in time sequences.

The power source unit 18 is a battery unit such as a button battery or capacitor, and is realized using a switch unit such as a magnetic switch. The power source unit 18 switches the on/off state of the power source using the magnetic field applied from the outside, and adequately supplies power of the battery unit to each component (imaging units 11A and 11B, wireless communication unit 16 and control unit 17) of the capsule endoscope 10 in case of the on state. Further, the power source unit 18 stops supplying power to each component of the capsule endoscope 10 in case of the off state.

The permanent magnet 19 enables the magnetic field generator 2 to magnetically guide the capsule endoscope 10. The permanent magnet 19 is fixed and arranged inside the capsule-shaped casing 12 in a state where the permanent magnet 19 is relatively fixed to the above imaging units 11A and 11B. In this case, the permanent magnet 19 magnetizes in a known, relatively fixed direction with respect to the up and down directions of imaging planes of the imaging elements 15A and 15B.

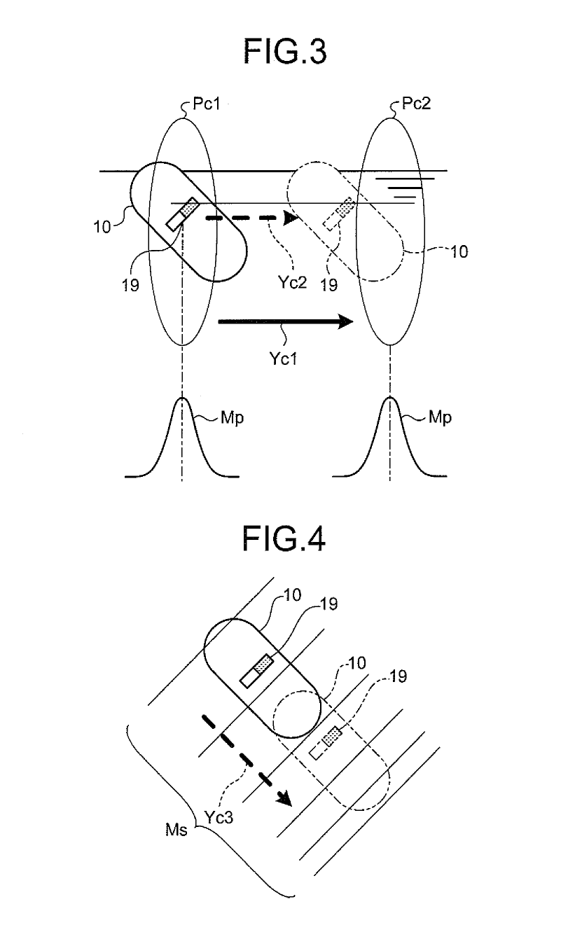

Next, the type of the magnetic field generated by the magnetic field generator 2 will be described. The magnetic field generator 2 can generate a so-called uniform magnetic field and, in addition, a peak magnetic field and uniform gradient magnetic field. As illustrated by the peak magnetic field Mp of FIG. 3, the peak magnetic field has a peak of a magnetic field intensity in a direction vertical to the horizontal plane. The peak magnetic field Mp can attract the permanent magnet 19 to the peak position of this magnetic field intensity to trap the capsule endoscope 10. That is, the peak magnetic field Mp attracts the permanent magnet 19 of the capsule endoscope 10 to an arbitrary position in the horizontal direction, and traps the capsule endoscope 10. By, for example, moving the peak position of the peak magnetic field Mp from a position Pc1 to a position Pc2 as indicated by an arrow Yc1, the magnetic field generator 2 can move the capsule endoscope 10 from the position Pc1 to the position Pc2 as indicated by an arrow Yc2.

Further, as illustrated by the uniform gradient magnetic field Ms in FIG. 4, the uniform gradient magnetic field has a nearly uniform magnetic gradient. This uniform gradient magnetic field biases the permanent magnet 19 in a direction in which a distribution of the magnetic field intensity becomes sparse to dense. By generating the uniform gradient magnetic field Ms in which the distribution of the magnetic field intensity becomes sparse to dense in a diagonally lower right direction from a diagonally upper left direction, the magnetic field generator 2 biases the permanent magnet 19 in a direction indicated by the arrow Yc3 to move the capsule endoscope 10 in the direction indicated by the arrow Yc3.

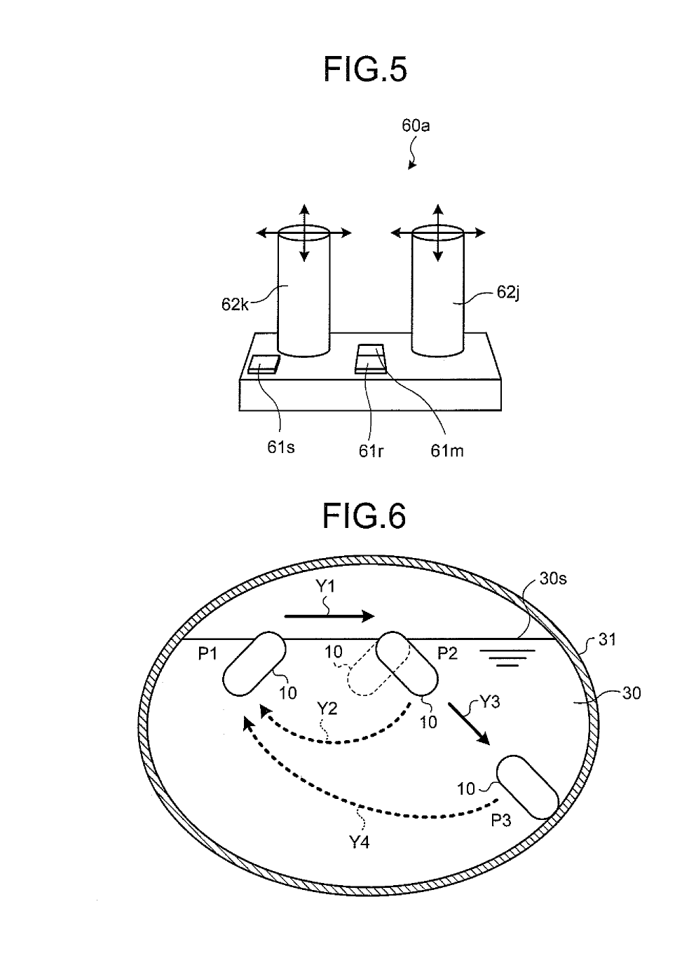

Next, the configuration of the operation input unit 60 will be described. FIG. 5 is a view illustrating an example of the operation input unit 60 illustrated in FIG. 1. An operation input unit 60a illustrated in this FIG. 5 is formed with, for example, a magnetic field switch button 61s, a mark setting button 61m, a mark return button 61r and two joy sticks 62j and 62k.

The magnetic field switch button 61s is pushed to input switch information for switching the type of the magnetic field generated by the magnetic field generator 2, to the external unit 4. The mark setting button 61m is pushed to input setting instruction information for instructing a setting of a mark in a guidance region of the capsule endoscope 10, to the external unit 4. The mark return button 61r is pushed to input return instruction information for instructing the capsule endoscope 10 to return to the mark set by pushing the mark setting button 61m. The joy sticks 62j and 62k can be operated to tilt in up and down directions and left and right directions, and are operated to tilt in the up and down directions and left and right directions to input to the external unit 4 operation information for performing a three-dimensional operation of the magnetic field generator 2 to magnetically guide the capsule endoscope 10.

When receiving an input of setting instruction information from the operation input unit 60, if the magnetic field control instruction unit 45 stores in the peak magnetic field storage unit 46 a generating condition of the peak magnetic field currently generated in the magnetic field generator 2, as position/posture information related to the position or posture of the capsule endoscope 10, the peak magnetic field traps the capsule endoscope 10 at a peak position of the magnetic field intensity with respect to the horizontal plane, so that storing the generating condition of this peak magnetic field corresponds to storing information of the position where the capsule endoscope 10 is currently positioned. In addition, if the peak magnetic field storage unit 46 has already stored the generating condition of the peak magnetic field as position/posture information, the peak magnetic field storage unit 46 updates position/posture information to the generating condition of the peak magnetic field which the peak magnetic field storage unit 46 is instructed to newly store.

Further, when receiving an input of return instruction information from the operation input unit 60, the magnetic field control instruction unit 45 makes the magnetic field generator 2 generate the magnetic field for guiding the capsule endoscope to the mark, based on position/posture information stored in the peak magnetic field storage unit 46. When receiving an input of return instruction information from the operation input unit 60, the magnetic field control instruction unit 45 reads the generating condition stored in the peak magnetic field storage unit 46, and makes the magnetic field generator 2 generate the peak magnetic field according to this generating condition. That is, the magnetic field control instruction unit 45 makes the magnetic field generator 2 generate the peak magnetic field of the same condition as the peak magnetic field generated upon an input of setting instruction information. As a result, the capsule endoscope 10 is guided to the same position as the position of the mark upon an input of setting instruction information, that is, upon pushing of the mark setting button 61m. In other words, the capsule endoscope 10 can return to the mark set by pushing the mark setting button 61m.

In addition, when the magnetic field switch button 61s inputs switch information, the magnetic field control instruction unit 45 instructs the magnetic field controller 8 to make the magnetic field generator 2 generate a uniform gradient magnetic field when the peak magnetic field is generated in the magnetic field generator 2, and instructs the magnetic field controller 8 to make the magnetic field generator 2 generate the uniform gradient magnetic field when the uniform gradient magnetic field is generated in the magnetic field generator 2.

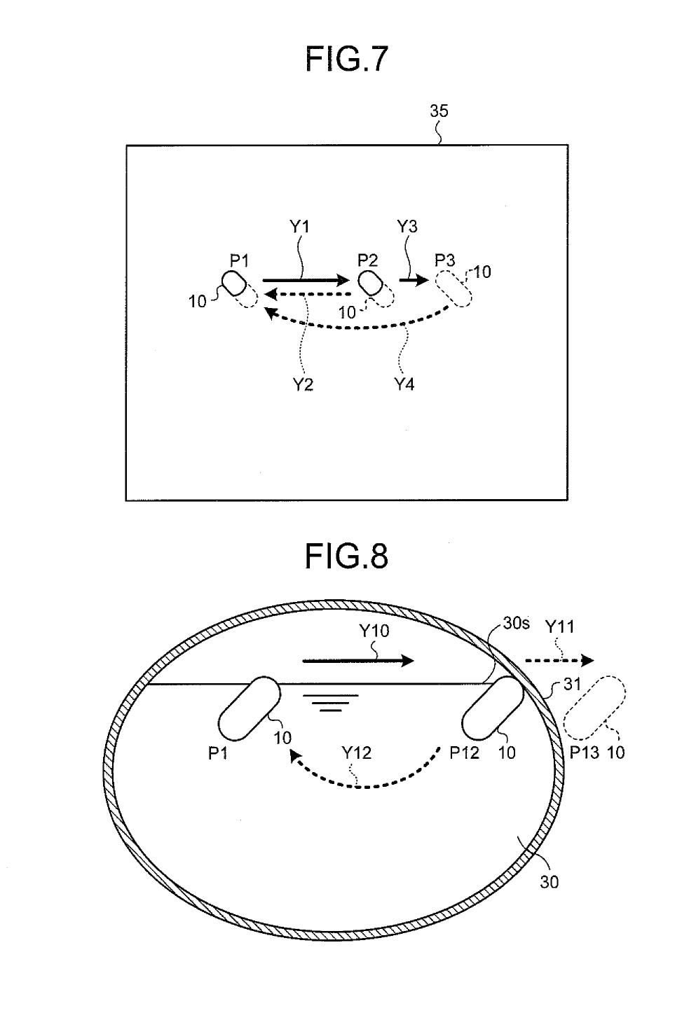

Next, as illustrated in FIG. 6 and FIG. 7, magnetic guidance of the capsule endoscope 10 by the operation of the operation input unit 60a will be described. FIG. 6 is a conceptual diagram for describing the state of the capsule endoscope 10 in a liquid 30 injected inside the stomach, illustrating the magnetic field generating area from the side. Further, FIG. 7 is a conceptual diagram for describing the state of the capsule endoscope 10 which moves the magnetic field generating area, illustrating a magnetic field generating area 35 from above.

First, a case will be described where the capsule endoscope 10 is guided inside a stomach 31 by the peak magnetic field. In this case, the operator operates the joy sticks 62j and 62k while checking images captured by the capsule endoscope 10 and displayed on the display unit 5 to guide the capsule endoscope 10 on a liquid level 30s. In this case, when, for example, a characteristic part of the organ which is medically characteristic is checked, if a position P1 of this characteristic part needs to be set as a mark, the mark setting button 61m is pushed. By this means, setting instruction information for instructing to set the mark is outputted to the external unit 4, and the generating condition of the peak magnetic field currently generated in the magnetic field generator 2, that is, the generating condition of the peak magnetic field for positioning the capsule endoscope 10 in the position P1 is stored in the peak magnetic field storage unit 46.

Then, the operator operates, for example, the joy sticks 62j and 62k to guide the capsule endoscope 10 to the position P2 at the liquid level as indicated by, for example, the arrow Y1 in FIG. 6 and FIG. 7. In this case, if the capsule endoscope 10 needs to be returned to the position P1 of the characteristic part set as the mark, the operator pushes the mark return button 61r. By this means, return instruction information is inputted to the external unit 4, and the magnetic field control instruction unit 45 generates the peak magnetic field according to the generating condition stored in the peak magnetic field storage unit 46. This peak magnetic field is generated according to a condition for positioning the capsule-endoscope 10 at the position P1, and therefore the capsule endoscope 10 is returned to the position P1 of the characteristic part set as the mark as indicated by the arrow Y2. As a result, the operator can continue observing the inside of the body from the characteristic part set as the mark.

Further, after moving the capsule endoscope 10 to a position P2, the operator pushes the magnetic field switch button 61s when the operator wants to observe the capsule endoscope 10 sunk in the liquid 30. As a result, switch information is inputted to the external unit 4, and the magnetic field control instruction unit 45 switches the magnetic field which the magnetic field control instruction unit 45 makes the magnetic field generator 2 generate, from the peak magnetic field to the uniform gradient magnetic field. Further, the magnetic field control instruction unit 45 makes the magnetic field generator 2 generate the uniform gradient magnetic field according to a condition matching operation information from the operation input unit 60. As a result, the capsule endoscope 10 submerges to, for example, the position P3 in the liquid 30 as indicated by the arrow Y3. In this case, if the capsule endoscope 10 needs to be returned to the position P1 of the characteristic part set as the mark, the operator pushes the mark return button 61r. By this means, return instruction information is outputted to the external unit 4, and the magnetic field control instruction unit 45 generates the peak magnetic field according to the generating condition stored in the peak magnetic field storage unit 46. As a result, as indicated by an arrow Y4, the capsule endoscope 10 returns to the position P1 of the characteristic part set as the mark, and the operator can continue observing the inside of the body from the characteristic part set as the mark. After the capsule endoscope 10 is returned to this mark, the magnetic field control instruction unit 45 may maintain or switch the magnetic field which the magnetic field control instruction unit 45 makes the magnetic field generator 2 generate as the peak magnetic field or to the uniform gradient magnetic field, according to operation information inputted from the operation input unit 60.

Thus, according to the first embodiment, when receiving an input of setting instruction information, the peak magnetic field storage unit 46 stores position/posture information related to the position or posture of the capsule endoscope 10 for setting the mark in the guidance region of the capsule endoscope 10, and, when receiving an input of return instruction information, the magnetic field control instruction unit 45 automatically returns the capsule endoscope 10 to the state where the mark is set by generating in the magnetic field generator 2 the magnetic field for guiding the capsule endoscope 10 to the mark based on position/posture information stored in the peak magnetic field storage unit 46.

Consequently, even when the operator sets the position of the mark by pushing the mark setting button 61m and therefore lost the sight of the capsule endoscope 10 while operating the operating unit and observing the internal organ, the operator can automatically return the capsule endoscope 10 to the position of the mark only by pushing the mark return button 61r, and can guide the capsule endoscope 10 and observe the inside of the body from the position where the relative relationship between the organ and the capsule endoscope 10 is clear. Consequently, according to the first embodiment, the operator does not need to perform a guiding operation to return the capsule-shaped medical device, so that it is possible to efficiently guide the capsule endoscope 10.

Further, as illustrated in FIG. 8, it is also effective when the capsule endoscope 10 moving on the liquid level 30s as indicated by the arrow Y10 according to the operation of the operator contacts the stomach wall at the position P12. In this case, when the operator performs an operation of moving the capsule endoscope 10 in the stomach 31 in the arrow Y11 direction without noticing that the capsule endoscope 10 contacts the stomach wall, even if the peak position of the peak magnetic field generated by the magnetic field generator 2 is displaced to the position P13 outside the stomach and the operator operates the operation input unit 60, there are cases where the capsule endoscope 10 does not move, and it is difficult to guide the capsule endoscope 10. Even in this case, if the operator pushes the mark setting button 61m in advance to set the mark, even when the position of the capsule endoscope 10 and the peak position of the peak magnetic field are displaced, thereby making it difficult to guide the capsule endoscope 10, it is possible to return the capsule endoscope 10 to the position P1 set as the mark as indicated by the arrow Y12 only by pushing the mark return button 61r. Consequently, even when it is difficult to guide the capsule endoscope 10, the operator can return the capsule endoscope 10 to a state where the capsule endoscope 10 can be guided immediately, only by pushing the mark return button 61r.

### Second Embodiment

Next, a second embodiment will be described. With the second embodiment, at least one of the position or posture of the capsule endoscope 10 is detected as posture information of the capsule endoscope 10, and this detection result is used as position/posture information.

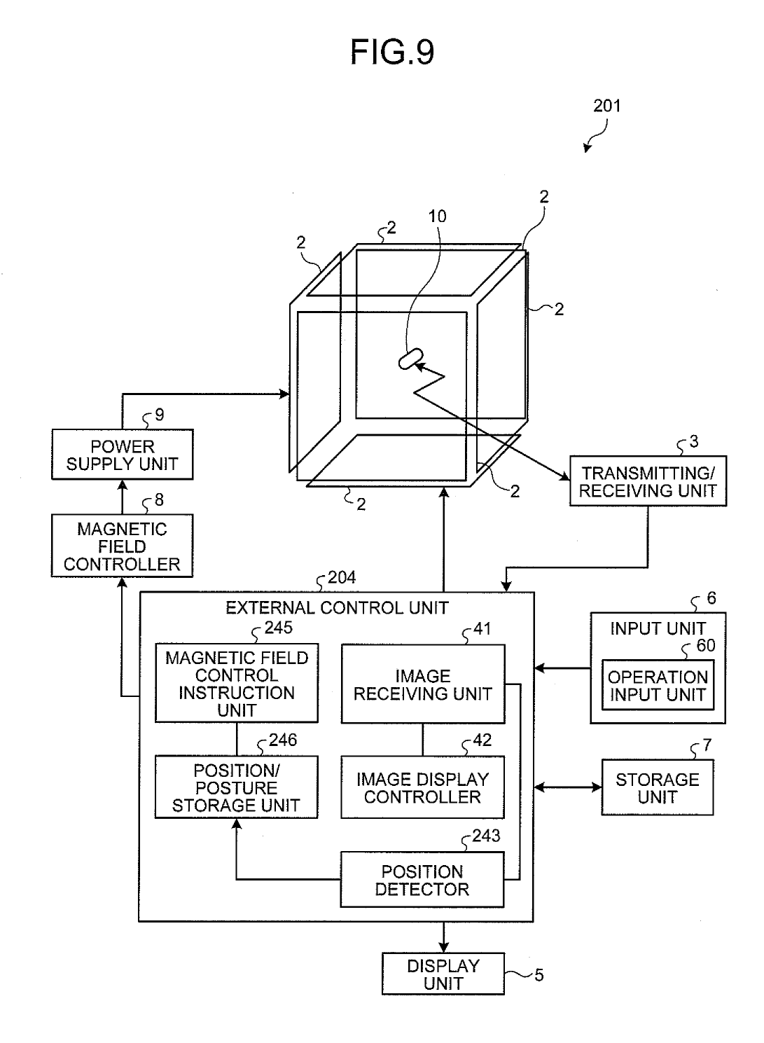

FIG. 9 is a schematic view illustrating an entire configuration of a capsule medical device guidance system according to the second embodiment. As illustrated in FIG. 9, a capsule medical device guidance system 201 according to the second embodiment employs a configuration including an external unit 204 instead of the external unit 4 illustrated in FIG. 1. The external unit 204 further includes a position detector 243 compared to the external unit 4 illustrated in FIG. 1. Further, compared to the external unit 4 illustrated in FIG. 1, the external unit 204 includes a magnetic field control instruction unit 245 and a position/posture storage unit 246 which stores position/posture information related to the position or posture of the capsule endoscope 10 instead of the magnetic field control instruction unit 45 and the peak magnetic field storage unit 46.

The position detector 243 detects at least one of the position and posture of the capsule medical device. The position detector 243 detects the position and posture of the capsule endoscope 10 inside the subject, based on the reception electric field intensity of the signal transmitted from the capsule endoscope 10. The position detector 243 calculates a position coordinate and direction vector of the capsule endoscope in the three-dimensional space. When receiving an input of setting instruction information from the operation input unit 60, the position detector 243 detects at least one of the position and posture of the capsule medical device.

The position/posture storage unit 246 stores the position or posture of the capsule endoscope 10 detected by the position detector 243 as position/posture information. In addition, when the position/posture storage unit 246 has already stored the detection result of the position detector 243 as position/posture information, the position/posture storage unit 246 updates position/posture information to a new detection result.

Similar to the magnetic field control instruction unit 45, when receiving an input of return instruction information from the operation input unit 60, the magnetic field control instruction unit 245 makes the magnetic field generator 2 generate the magnetic field for guiding the capsule endoscope 10 to the mark based on position/posture information stored in the position/posture storage unit 246. When receiving an input of return instruction information from the operation input unit 60, the magnetic field control instruction unit 245 reads the position or posture of the capsule endoscope 10 stored in the position/posture storage unit 246, and controls the magnetic field generator 2 such that the capsule endoscope 10 takes this position or posture. In this case, the capsule endoscope 10 is guided to take the position or posture stored in the position/posture storage unit 246, and therefore the magnetic field control instruction unit 245 makes magnetic field generator 2 generate the peak magnetic field or uniform gradient magnetic field according to a condition matching the position or posture stored in the position/posture storage unit 246.

Thus, according to the second embodiment, when receiving an input of setting instruction information, the position/posture storage unit 246 stores the position or posture of the capsule endoscope 10 detected by the position detector 243 to set the mark, in the guidance region of the capsule endoscope 10, and, when receiving an input of return instruction information, the magnetic field control instruction unit 245 makes the magnetic field generator 2 generate the magnetic field such that the capsule endoscope 10 takes the position or posture stored in the position/posture storage unit 246, and automatically returns the capsule medical device to a state where the mark is set. Consequently, according to the second embodiment, the operator does not need to perform a guiding operation to return the capsule medical device similar to the first embodiment, so that it is possible to efficiently guide the capsule endoscope 10.

In addition, the position detector 243 may detect the center position of the peak of the peak magnetic field generated by the magnetic field generator 2 upon an input of setting instruction information, or may detect the position or posture of the capsule endoscope 10 upon an input of setting instruction information based on the direction of the magnetic field generated around the peak. That is, the position detector 243 may detect the position or posture of the capsule endoscope 10 upon an input of setting instruction information, based on the position which attracts the permanent magnet 19 of the peak magnetic field generated by the magnetic field generator 2, or the direction of the magnetic field generated at the position which attracts the permanent magnet 19. Further, the position detector 243 may detect both of the position and posture of the capsule endoscope 10.

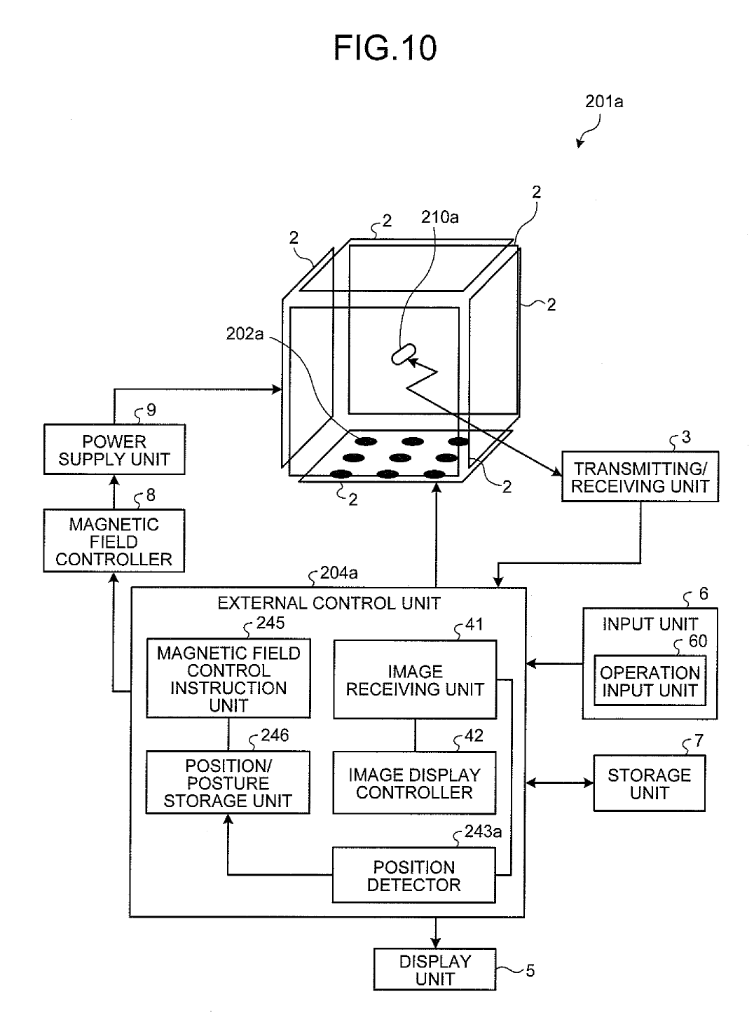

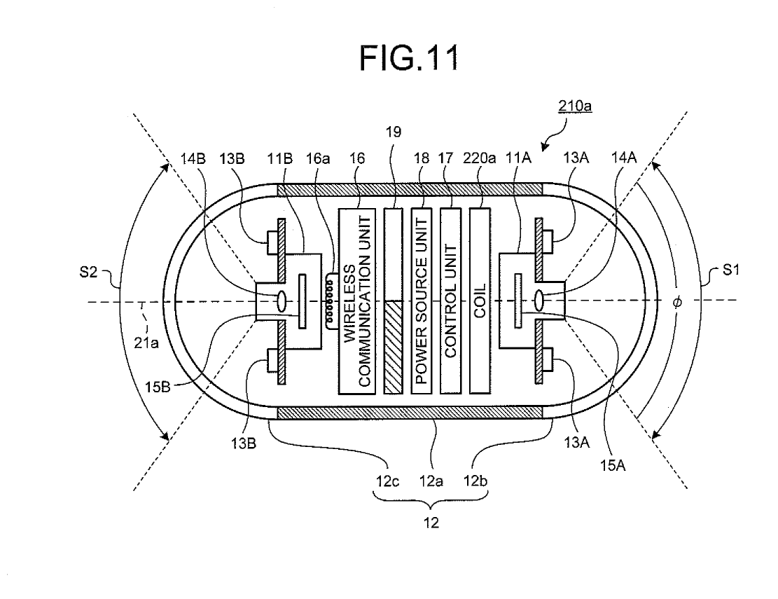

Further, as illustrated in FIG. 10 and FIG. 11, the capsule medical device guidance system according to the second embodiment may be a capsule medical device guidance system 201a which uses a capsule endoscope 210a further including coils 220a which generates an alternate current magnetic field, and which has outside the capsule endoscope 210a a magnetic field detecting unit 202a which detects the alternate current magnetic field generated by the capsule endoscope 210a formed with a plurality of magnetic field detecting coils. In this case, a position detector 243a of an external unit 204a calculates a position coordinate and direction vector of the capsule endoscope 210a in the three-dimensional space, based on the detection result of the magnetic field detecting unit 202a.

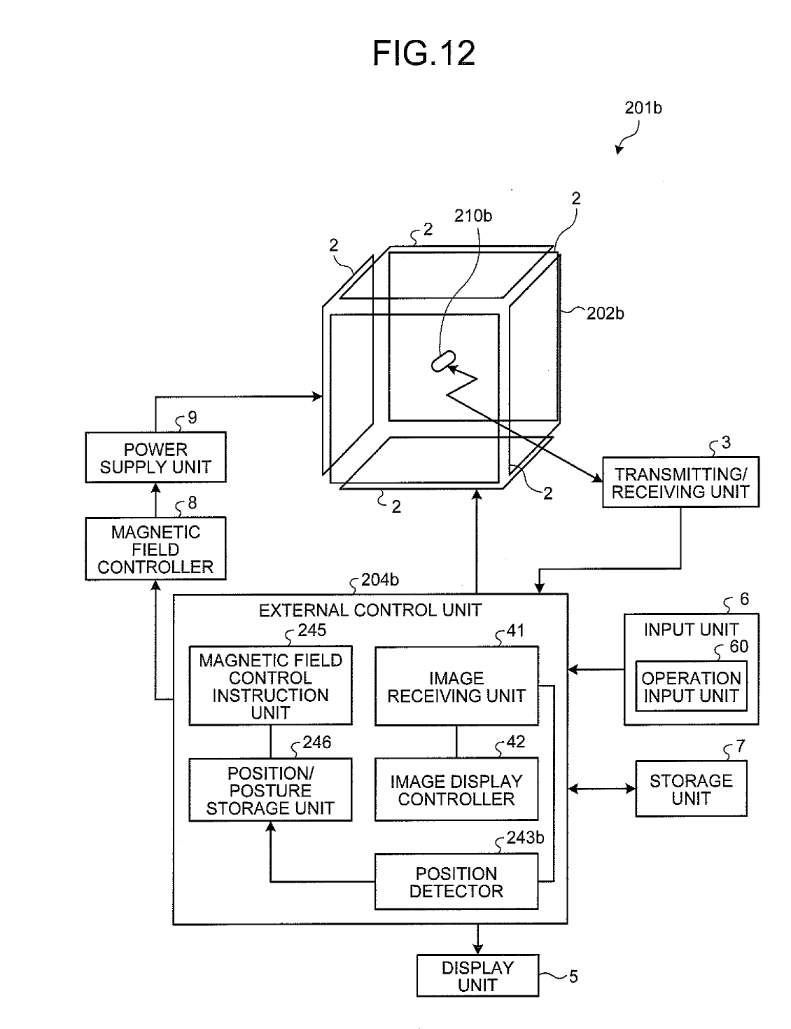

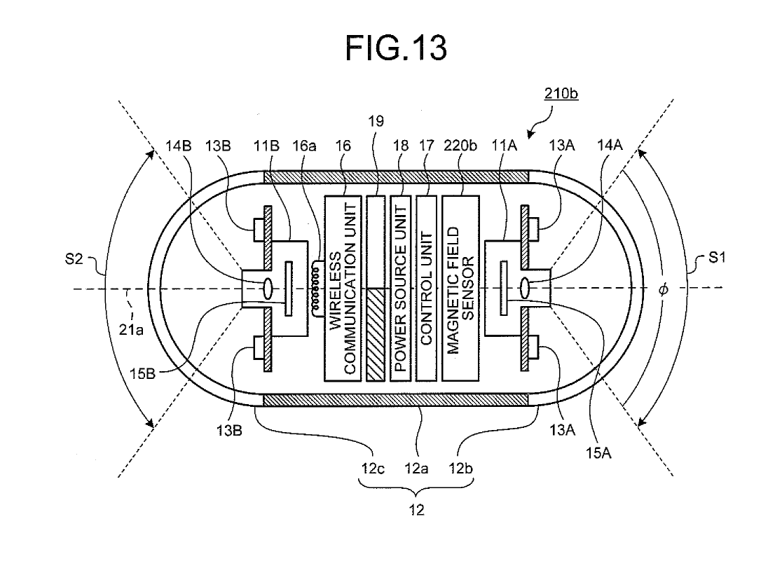

Further, as illustrated in FIG. 12 and FIG. 13, the capsule medical device guidance system according to the second embodiment may be a capsule medical device guidance system 201b which has a position detecting magnetic field generator 202b which generates an alternate current magnetic field for position detection in a capsule endoscope 210b formed with a plurality of coils which generate an alternate current, and further includes a magnetic field sensor 220b which detects the alternate current magnetic field, and which uses the capsule endoscope 210b which transmits the detection result of the magnetic field sensor 220b to the transmitting/receiving unit 3. In this case, a position detector 243b of an external unit 204b calculates the position coordinate and direction vector of the capsule endoscope 210b in the three-dimensional space, based on the detection result of the alternate current magnetic field in the capsule endoscope 210b received in the transmitting/receiving unit 3.

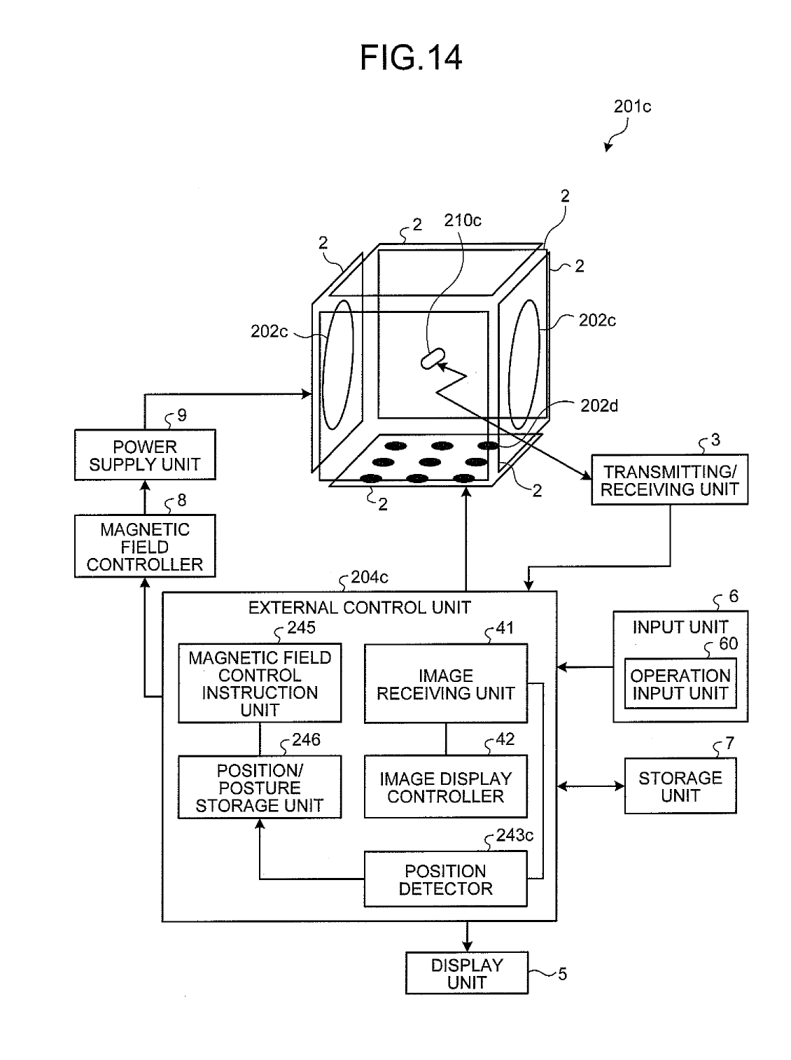

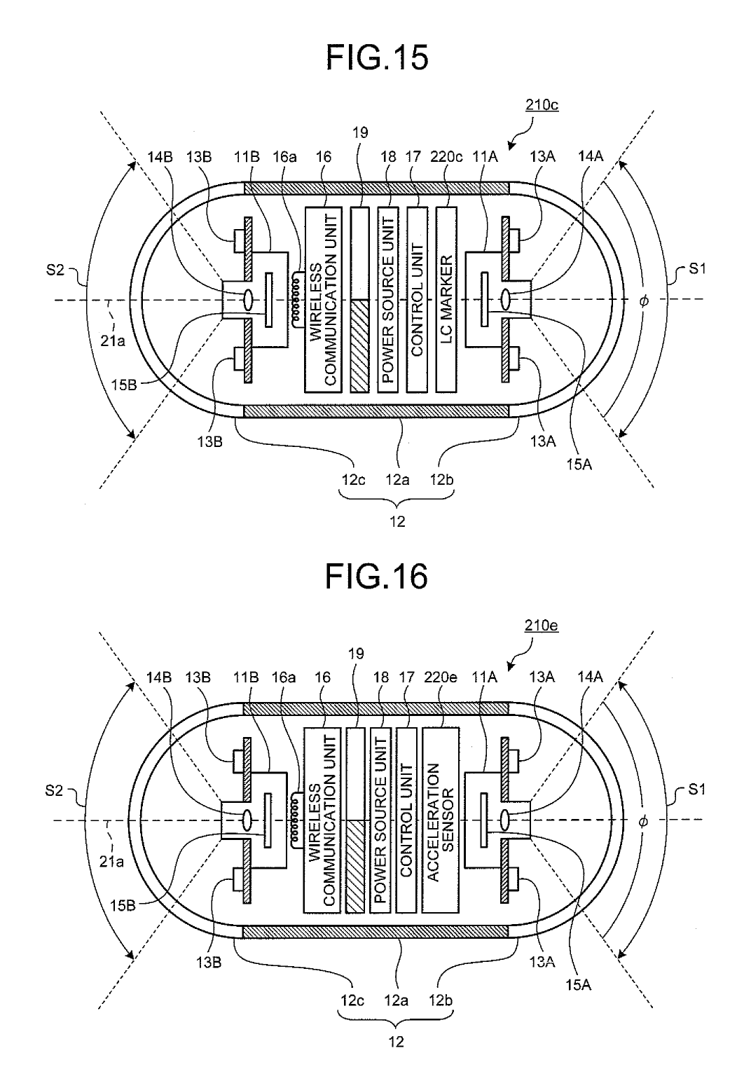

Further, as illustrated in FIG. 14 and FIG. 15, the capsule medical device guidance system according to the second embodiment may be a capsule medical device guidance system 201c which uses a capsule endoscope 210c further including a LC marker 220c, and which includes outside the capsule endoscope 210c a position detecting magnetic field generator 202c which generates an alternate current for position detection in the capsule endoscope 210c formed with a plurality of coils which generate an alternate magnetic field, and a magnetic field detecting unit 202d which detects a guidance magnetic field generated by the LC marker 220c. In this case, a position detector 243c of an external unit 204c calculates a position coordinate and direction vector of the capsule endoscope 210c in the three-dimensional space, based on the detection result of the magnetic field detecting unit 202d.

Further, as illustrated in FIG. 16, the capsule medical device guidance system according to the second embodiment may use a capsule endoscope 210e which further includes an acceleration sensor 220e and transmits to the transmitting/receiving unit 3 an output result of the acceleration sensor 220e. In this case, the position detector 243 detects a relative change amount of the position and posture of the capsule endoscope 210e by integrating output results of the acceleration sensor 220e transmitted from the capsule endoscope 210e, and detects the position and posture of the capsule endoscope 210e.

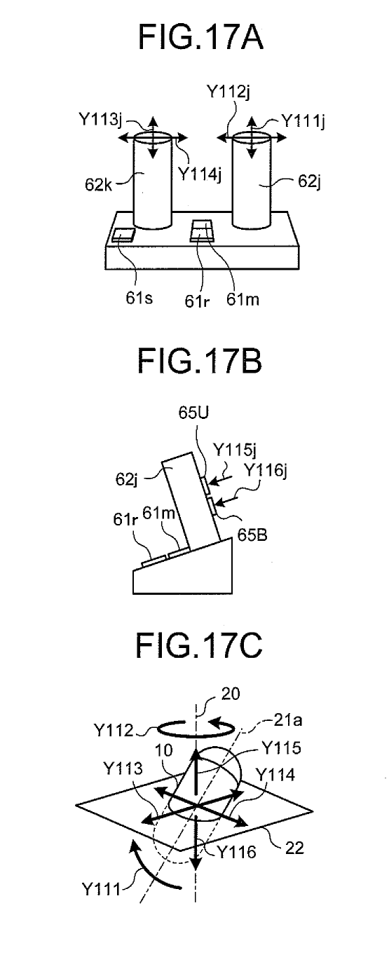

Further, the motion of the capsule endoscope 10 matching the guiding operation of the operation input unit 60a illustrated in FIG. 5 will be described. FIG. 17A is a front view of an operation input unit for describing magnetic guidance of a capsule medical device which can be operated by the operation input unit. FIG. 17B is a right side view of an operation input unit for describing magnetic guidance of a capsule medical device which can be operated by the operation input unit. FIG. 17C is a view illustrating content of an operation of a capsule endoscope instructed by an operation of each component of the operation input unit.

As illustrated in FIG. 17A, a tilting direction of the joy stick 62j in the up and down directions indicated by an arrow Y111j corresponds to a tilting operation direction in which the distal end of the capsule endoscope 10 oscillates to pass a vertical axis 20 as indicated by an arrow Y111 in FIG. 17C. When the operation input unit 60a inputs to the external unit 4 operation information matching the tilting operation of the joy stick 62j indicated by the arrow Y111j, the magnetic field control instruction unit 45 computes a guiding direction of the distal end of the capsule endoscope 10 on the absolute coordinate, based on this operation information according to the tilting direction of the joy stick 62j and computes a guiding speed according to the tilting operation of the joy stick 62j. Further, the magnetic field control instruction unit 45 makes magnetic field generator 2 generate the peak magnetic field having, for example, an orientation matching the computed guiding direction, and changes an angle formed between the orientation of this peak magnetic field and the vertical axis 20 at the computed guiding speed, in the vertical plane including the vertical axis 20 and the long axis 21a of the capsule endoscope 10.

As illustrated in FIG. 17A, the tilting direction of the joy stick 62j in the left and right directions indicated by an arrow Y112j matches a rotation operation direction in which the capsule endoscope 10 rotates about the vertical axis 20 as indicated by the arrow Y112 in FIG. 17C. When the operation input unit 60a inputs to the external unit 4 operation information matching the tilting operation of the joy stick 62j indicated by the arrow Y112j, the magnetic field control instruction unit 45 computes the guiding direction of the distal end of the capsule endoscope 10 on the absolute coordinate system according to the tilting direction of the joy stick 62j, computes the guiding speed according to the tilting operation of the joy stick 62j, makes the magnetic field generator 2 generate the peak magnetic field having, for example, an orientation matching the computed guiding direction, and rotates and moves the orientation of this peak magnetic field about the vertical axis 20 at the computed guiding speed.

As illustrated in FIG. 17A, the tilting direction of the joy stick 62k in the up and down directions indicated by an arrow Y113j matches a horizontal backward operation direction or horizontal forward operation direction in which the capsule endoscope 10 travels toward a direction in which the long axis 21a is projected on a horizontal plane 22 as indicated by the arrow Y113 in FIG. 17C. When the operation input unit 60a inputs to the external unit 4 operation information matching the tilting operation of the joy stick 62k indicated by the arrow Y113j, the magnetic field control instruction unit 45 computes the guiding direction and guiding position of the distal end of the capsule endoscope 10 on the absolute coordinate system, based on this operation information according to the tilting direction of the joy stick 62k, computes the guiding speed according to the tilting operation of the joy stick 62k, makes the magnetic field generator 2 generate the peak magnetic field having, for example, an orientation matching the computed guiding direction, and moves the peak of this peak magnetic field to the guiding position at the computed guiding speed.

As illustrated in FIG. 17A, the tilting direction of the joy stick 62k in the left and right directions indicated by an arrow Y114j matches a horizontal right operation direction or horizontal left operation direction in which the capsule endoscope 10 travels on the horizontal plane 22 vertically to a direction in which the long axis 21a is projected on the horizontal plane 22 as indicated by the arrow Y114 in FIG. 17C. When the operation input unit 60a inputs to the external unit 4 operation information matching the tilting operation of the joy stick 62k indicated by the arrow Y114j, the magnetic field control instruction unit 45 computes the guiding direction and guiding position of the distal end of the capsule endoscope 10 on the absolute coordinate system, based on this operation information according to the tilting direction of the joy stick 62k, computes the guiding speed according to the tilting operation of the joy stick 62k, makes the magnetic field generator 2 generate the peak magnetic field having, for example, an orientation matching the computed guiding direction, and moves the peak of this peak magnetic field to the guiding position at the computed guiding speed.

Further, an up button 65U and a down button 65B are provided in the back surface of the joy stick 62k. When the up button 65U is pushed as indicated by an arrow Y115J in FIG. 17B, an up operation is instructed such that the capsule endoscope 10 moves upward along the vertical axis 20 illustrated in FIG. 17C as indicated by the arrow Y115. Further, as indicated by an arrow 116j in FIG. 17B, when the down button 65B is pushed, a down operation is instructed such that the capsule endoscope 10 moves downward along the vertical axis 20 illustrated in FIG. 17C as indicated by an arrow 116. When the operation input unit 60a inputs to the external unit 4 operation information matching a pushing operation of the up button 65U or the down button 65B indicated by the arrow Y115j and Y116j, the magnetic field control instruction unit 45 computes the operation direction of the distal end of the capsule endoscope 10 on the absolute coordinate system, based on this operation information according to which one of the buttons is pushed, and makes the magnetic field generator 2 generate a uniform gradient magnetic field having a gradient along the vertical axis 20 according to the computed operation direction. When the up button 65U is pushed, the magnetic field generator 2 generates a uniform gradient magnetic field having a gradient which becomes dense toward the upper direction of the vertical axis 20 to move the capsule endoscope 10 as indicated by the arrow Y115. When the down button 65B is pushed, the magnetic field generator 2 generates a uniform gradient magnetic field having a gradient which becomes dense toward the lower direction of the vertical axis 20 to move the capsule endoscope 10 as indicated by the arrow Y116.

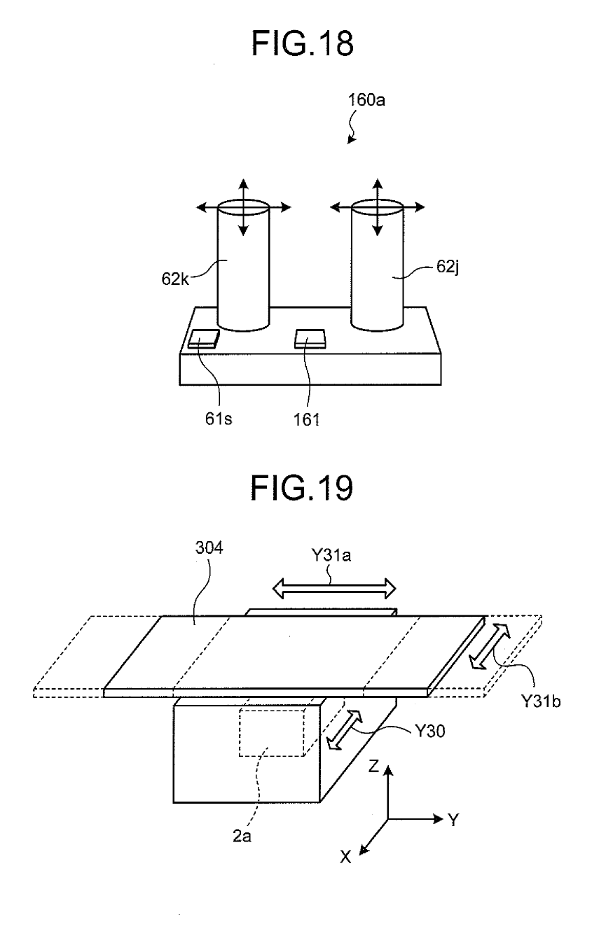

Further, although the operation input unit 60a which has the mark setting button 61m and the mark return button 61r has been described in FIG. 5 as an example of the operation input unit 60 illustrated in FIG. 1, the operation input unit is not limited to this, and, as illustrated in FIG. 18, may be an operation input unit 160a which has a mark button 161 which can instruct both of a setting of the mark and return of the mark. In this case, the mark button 161 inputs to the external unit 4 setting instruction information when a pushing time is equal to or greater than a predetermined time, and inputs to the external unit 4 return instruction information when the pushing time is less than a predetermined time. In addition, the mark button 161 inputs to the external unit 4 setting instruction information when the mark button 161 is pushed twice, and inputs to the external unit 4 return instruction information when the mark button 161 is pushed once. Thus, instruction information to be inputted to the external unit 4 may be identified by changing an input method of one mark button 161.

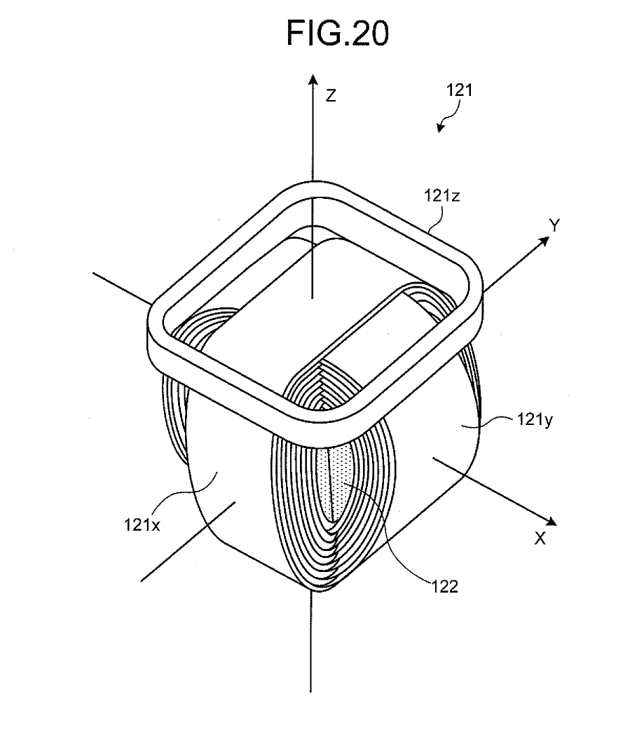

Further, the magnetic field generator 2 according to the first and second embodiments changes relative positions of a bed 304 which supports the patient who is the subject, and a magnetic field generator 2a which generates the peak magnetic field on the center axis to generate a peak magnetic field having a peak at a desired position inside the subject. FIG. 19 is a schematic view illustrating an example of each movement state of a table part of a bed 304 and magnetic field generator. As illustrated in FIG. 19, the bed 304 can be moved horizontally in a Y axis direction of the absolute coordinate system as indicated by an arrow Y31a, and the magnetic field generator 2a can be moved horizontally in the X axis direction of the absolute coordinate system as indicated by the arrow Y30. In this case, by moving the bed 304 and the magnetic field generator 2a, the relative positions of the bed 304 and the magnetic field generator 2a are changed and the peak magnetic field having a peak at a predetermined position is generated on the horizontal plane. Further, when the bed 304 can be moved in the X axis direction of the absolute coordinate system as indicated by the arrow Y31b as well as in the Y axis direction of the absolute coordinate system, the relative positions of the bed 304 and the magnetic field generator 2a may be changed by moving only the bed 304. Further, when the magnetic field generator 2a can be moved in the Y axis direction of the absolute coordinate system in addition to the X axis direction of the absolute coordinate system, the relative positions of the bed 304 and the magnetic field generator 2a may be changed by moving only the magnetic field generator 2a.

Further, the magnetic field generator 2a generates a guidance magnetic field by three-dimensionally combining three axial direction coils which generate, for example, magnetic fields in each axis direction of the absolute coordinate system. FIG. 20 is a schematic view illustrating an example of the magnetic field generator illustrated in FIG. 19. As illustrated in FIG. 20, like a magnetic field generator 121, the magnetic field generator according to the present invention is realized by three-dimensionally combining a X axis coil 121x which generates the magnetic field in the X axis direction of the absolute coordinate system, a Y axis coil 121y which generates the magnetic field in the Y axis direction of the absolute coordinate system and a Z axis coil 121z which generates the magnetic field in the Z axis direction of the absolute coordinate system. The X axis coil 121x and Y axis coil 121y wind around an iron core 122 orthogonally to each other. The Z axis coil 121z is arranged above the X axis coil 121x and Y axis coil 121y.

Further, although cases have been described as examples with the first and second embodiments where the capsule endoscope 10 including a plurality of imaging units is used, it naturally follows that a capsule endoscope including only the imaging unit 11A may be used.

Further, although cases have been described as examples with the first and second embodiments where the capsule endoscope 10 using the permanent magnet 19 is used, it naturally follows that the capsule endoscope is not limited to this and may have an electrical magnet instead of the permanent magnet 19.

Further, the peak magnetic field storage unit 46 and the position/posture storage unit 246 may store a plurality of pieces of position/posture information, and the magnetic field control instruction units 45 and 245 may control the magnetic field generator 2 to return the capsule endoscope 10 from a new mark back to an old mark tracing back in order of the stored latest position/posture information based on the number of times to push the mark return button 61r.

Further, the operation input unit 160a may have a plurality of mark buttons 161, the peak magnetic field storage unit 46 and the position/posture storage unit 246 store a plurality pieces of position/posture information in association with a setting operation to each mark button 161, and the magnetic field control instruction units 45 and 245 may control the magnetic field generator 2 such that the capsule endoscope 10 returns to the instructed mark, based on position/posture information which is stored in the peak magnetic field storage unit 46 and the position/posture storage unit 246 and which is related to the mark button 161 to which a return operation is inputted.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A capsule medical device guidance system comprising:
a capsule medical device to be introduced into a subject, the capsule medical device including a magnetic field responding unit;
a magnetic field generator that generates a magnetic field for the magnetic field responding unit to guide the capsule medical device;
an operation input unit for inputting operation information for magnetically guiding the capsule medical device;
a storage unit that stores position/posture information about a position or a posture of the capsule medical device, in an guidance region in which the magnetic field generator allows the capsule medical device to be guided;
an instruction information input unit for inputting setting instruction information for setting the position/posture information as a mark indicating a position to which the capsule medical device is returned in the guidance region, and return instruction information for returning the capsule medical device to the mark; and
a control unit that controls the magnetic field generator to guide the capsule medical device in accordance with the operation information input through the operation input unit, and makes the magnetic field generator generate a magnetic field for guiding the capsule medical device to the mark based on position/posture information stored in the storage unit when the return instruction information is input through the instruction information input unit.

2. The capsule medical device guidance system according to claim 1, wherein
the magnetic field generator generates a trapping magnetic field for trapping the capsule medical device so that the magnetic field responding unit is attracted to any position in a horizontal plane in a space into which the capsule medical device is guided,
the storage unit stores, as the position/posture information, a generating condition that the trapping magnetic field is generated by the magnetic field generator when the setting instruction information is input through the instruction information input unit, and
the control unit makes the magnetic field generator generate the trapping magnetic field under the generating condition stored in the storage unit when the return instruction information is input through the instruction information input unit.

3. The capsule medical device system according to claim 1, further comprising a detecting unit that detects at least one of a position and a posture of the capsule medical device, wherein
the detecting unit detects the at least one of the position and the posture of the capsule medical device when the setting instruction information is input through the instruction information input unit,
the storage unit stores the position or the posture of the capsule medical device detected by the detecting unit as the position/posture information, and
the control unit controls the magnetic field generator so that the capsule medical device is at the position or the posture stored in the storage unit when the return instruction information is input through the instruction information input unit.

4. The capsule medical device guidance system according to claim 3, wherein
the magnetic field generator generates a trapping magnetic field for trapping the capsule medical device so that the magnetic field responding unit is attracted to any position in a horizontal plane in a space into which the capsule medical device is guided, and
the detecting unit detects at least one of a position and a posture of the capsule medical device based on either a position to which the trapping magnetic field generated by the magnetic field generator attracts the magnetic field responding unit or a direction of a magnetic field generated at a position to which the magnetic field responding unit is attracted.
